(19) United States
(12) Patent Application Publication
(10) Pub. No.: US 2005/0148069 A1
(43) Pub. Date: Jul. 7, 2005

(54) IGF-1 instructs multipotent adult CNS neural stem cells to an oligodendroglial lineage

(76) Inventors: Fred H. Gage, La Jolla, CA (US); Jenny Hsieh, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/949,017

(22) Filed: Sep. 24, 2004

Related U.S. Application Data
(60) Provisional application No. 60/505,984, filed on Sep. 24, 2003.

(51) Int. Cl. C12N 5/08

(52) U.S. Cl. 435/366; 435/368

(57) ABSTRACT

Adult neural stem cells differentiate into neurons, astrocytes, and oligodendrocytes in the mammalian CNS, but the molecular mechanisms that control their differentiation are not yet well understood. Insulin-like growth factor-I (IGF-I) can promote the differentiation of cells already committed to an oligodendroglial lineage during development. However, it is unclear whether IGF-I affects multipotent neural stem cells. Here we show that IGF-I stimulates the differentiation of multipotent adult rat hippocampus-derived neural progenitor cells into oligodendrocytes. Modeling analysis indicates that the actions of IGF-I are instructive. Oligodendrocyte differentiation by IGF-I appears to be mediated through an inhibition of BMP signaling. Furthermore, overexpression of IGF-I in the hippocampus leads to an increase in oligodendrocyte markers. These data demonstrate the existence of a single molecule, IGF-I, that can influence the fate choice of multipotent adult neural progenitor cells to an oligodendroglial lineage.

### CROSS-REFERENCES TO RELATED APPLICATIONS

The present application claims priority to U.S. Ser. No. 60/505,984, filed Sep. 24, 2003, herein incorporated by reference in its entirety.

### STATEMENT AS TO RIGHTS TO INVENTIONS MADE UNDER FEDERALLY SPONSORED RESEARCH AND DEVELOPMENT

This invention was made with government support under Grant No. ______, awarded by the NIH. The government has certain rights in this invention.

### REFERENCE TO A “SEQUENCE LISTING,” A TABLE, OR A COMPUTER PROGRAM LISTING APPENDIX SUBMITTED ON A COMPACT DISK.

Not applicable.

### FIELD OF THE INVENTION

Adult neural stem cells differentiate into neurons, astrocytes, and oligodendrocytes in the mammalian CNS, but the molecular mechanisms that control their differentiation are not yet well understood. Insulin-like growth factor-I (IGF-I) can promote the differentiation of cells already committed to an oligodendroglial lineage during development. However, it is unclear whether IGF-I affects multipotent neural stem cells. Here we show that IGF-I stimulates the differentiation of multipotent adult rat hippocampus-derived neural progenitor cells into oligodendrocytes. Modeling analysis indicates that the actions of IGF-I are instructive. Oligodendrocyte differentiation by IGF-I appears to be mediated through an inhibition of BMP signaling. Furthermore, overexpression of IGF-I in the hippocampus leads to an increase in oligodendrocyte markers. These data demonstrate the existence of a single molecule, IGF-I, that can influence the fate choice of multipotent adult neural progenitor cells to an oligodendroglial lineage.

### BACKGROUND OF THE INVENTION

Multipotent cells with the ability to divide and differentiate have been shown to exist throughout the central nervous system (CNS), yet neurogenesis appears to be restricted to two specific brain regions within the adult CNS: the subventricular zone and the hippocampal subgranular zone (Gage, 2000). Beyond these two regions, most of the dividing cells in other areas give rise to new glial cells and not neurons (Homer et al., 2000; Komack and Rakic, 2001; Rakic, 2002). However, when these dividing cells are isolated from different regions of the CNS (Lois and Alvarez-Buylla, 1993; Palmer et al., 1997; Reynolds and Weiss, 1992), including non-neurogenic areas (Kondo and Raff, 2000; Palmer et al., 1999; Shihabuddin et al., 1997), they all retain the ability to self-renew and differentiate into neurons, oligodendrocytes and astrocytes in culture. Remarkably, even proliferating cells isolated from non-neurogenic areas can differentiate into neurons when transplanted back into the hippocampus (Shihabuddin et al., 2000; Suhonen et al., 1996), suggesting that cues from the local environment influence their fate determination and differentiation programs.

Recent work by Song and colleagues (Song et al., 2002) directly examined the idea that different environments can influence the fate specification of neural stem cells. Astrocytes from neurogenic regions (but not non-neurogenic regions) can preferentially direct multipotent adult neural stem cells to differentiate into neurons. Interestingly, these neural stem cells appear to preferentially differentiate into oligodendrocytes when cultured together with hippocampus-derived neurons. These results suggest that specific signals from different CNS environments exist for lineage-specific differentiation. Examples of extrinsic factors important for neuronal and astroglial differentiation of multipotent adult neural stem cells have already been identified (Lim et al., 2000; Takahashi et al., 1999; Tanigaki et al., 2001). However, little is known regarding the control of oligodendrocyte differentiation of multipotent adult neural stem cells.

Insulin-like growth factors (IGFs) comprise one class of molecules that have effects on oligodendrocyte biology during development (D'Ercole et al., 1996). IGFs (IGF-I, IGF-II) and insulin can all independently promote the survival of purified oligodendrocytes in culture (Barres et al., 1993). Furthermore, IGFs have important roles in the proliferation and differentiation of cells that have already committed to an oligodendroglial lineage during development (McMorris and Dubois-Dalcq, 1988; McMorris et al., 1986). Furthermore, transgenic and knockout mouse models have revealed in vivo effects of IGFs on oligodendrocyte development and myelination. Overexpression of IGF-I in transgenic mice results in increased brain size and myelin content (Carson et al., 1993; Ye et al., 1995). Conversely, IGF-I knockout mice have smaller brains, reduced oligodendrocyte numbers, and CNS hypomyelination (Baker et al., 1993; Beck et al., 1995; Carson et al., 1993; Ye et al., 2002). In addition, the following patents are related to differentiation of neural stem cells and/or the role of IGF-I in this process: U.S. 20040009593; U.S. Pat. No. 5,750,376; U.S. 20030054551; U.S. 20030049838. It has been suggested that IGF-1 plays a role in CNS disease states (see, e.g., Kaspar et al., Science 301:839-842 (20030; Mason et al., J. Neurosci. 23:7710-7718 (2003); and U.S. patent publication Nos. 20030118556 and 20030118552). Finally, neurogenesis, e.g., hippocampal neurogenesis, has also been implicated in CNS disease states (see, e.g., Gage, Scientific American, September 2003, 47-53; Tkachev et al., The Lancet 362:798-805 (2003); Santarelli et al, Science 301:805-809 (2003)).

### BRIEF SUMMARY OF THE INVENTION

With this invention, we show that IGF-I can preferentially induce the differentiation of multipotent adult neural progenitor cells into oligodendrocytes. Using a modeling approach, we show that the IGF-I-induced increase in oligodendrocyte numbers is attributable to an instructive differentiation of uncommitted cells to an oligodendroglial fate, not to a selective proliferation or survival of committed oligodendrocyte progenitors. The IGF-I-induced oligodendrocyte differentiation appears, at least in part, to be mediated by the inhibition of bone morphogenetic protein (BMP) signaling. Furthermore, overexpression of IGF-I in the hippocampus led to an increase in immunoreactivity for oligodendrocyte markers. These results provide evidence that IGF-I is an important regulator of oligodendrocyte differentiation from multipotent adult neural progenitor cells. Therefore, stimulation of the IGF-1 receptor with IGF-1 or another growth factor or compound that binds to the IGF-1 receptor, such as insulin and/or IGF-2, can be used to stimulate differentiation of adult neural stem cells into oligodendrocytes. Currently the lack of markers to distinguish a multipotent neural stem cell from a multipotent progenitor cell has prompted us to call our cell culture system multipotent adult neural progenitor cells.

### BRIEF DESCRIPTION OF THE DRAWINGS

FIG. 1. IGF-I induced differentiation of multipotent neural progenitor cells. (A) Adult hippocampus-derived neural progenitor cells cultured in 20 ng/ml FGF-2 (undifferentiated), 1 μM RA and 1% FBS for 4 days (mixed), 1 μM RA and 5 μM FSK for 4 days (neuronal), or 50 ng/ml LIF and 50 ng/ml BMP2 for 6 days (astrocytic). Cells were stained for markers for neurons (Tuj1), astrocytes (GFAP), or oligodendrocytes (RIP). Scale bar, 50 μM. DAPI, 4,6-diamidino-2-phenylindole. (B) Differentiation of neural progenitor cells in insulin-free media without IGF-I (control) or with IGF-I for 4 days. Enlarged inserts show cells of typical oligodendrocyte morphology stained with RIP or MBP. Scale bar, 25 μM. (C) Quantification of cells in either proliferating or differentiating conditions. (D) Quantification of cultures grown in insulin-free media (control) or treated with 500 ng/ml IGF-I, IGF-II, or insulin after 4 days. (E) Quantification of cells in response to different doses of IGF-I (20-500 ng/ml) in 4-day cultures. All data shown are from at least 3 experiments in parallel cultures with error bars representing standard deviations. Significant differences are indicated with an asterisk (p<0.005).

FIG. 2. IGF-I-mediated increase in oligodendrocyte differentiation is independent of effects on neural progenitor cell survival in 2-day cultures. (A) Neural progenitors cultured in insulin-free N2 medium (control) exhibited massive cell death, as shown by fragmented DAPI-stained nuclei. (B) Treatment of cells with 2 μM Q-VD-OPh resulted in a general absence of apoptosis in short-term cultures. The maintenance of cell survival is shown by a general persistence of cells and lack of fragmented DAPI-stained nuclei, without obvious effects on cell proliferation or differentiation. (C) Addition of 500 ng/ml IGF-I to the Q-VD-OPh-treated cultures mediated an increase in oligodendrocyte differentiation. (D) Quantification of RIP+ oligodendrocytes. Scale bar, 50 μM.

FIG. 3. Effect of IGF-I on the survival, proliferation, and instructive oligodendrocyte differentiation of adult neural progenitor cells. (A) Quantification of cell death in insulin-free (no IGF-I) and IGF-I-treated (500 ng/ml) cultures, as determined every 12 hours by live staining with propidium iodide (1 μg/ml) and Hoechst 33342 (1 μg/ml). (B) Proliferation in insulin-free (no IGF-I) and IGF-I-treated (500 ng/ml) cultures as determined by BrdU incorporation. Parallel cultures were incubated with BrdU (2.5 μM) at the beginning of each 12-h time point, immediately followed by fixation and BrdU staining. BrdU+ cells were counted as either being RIP−(blue and black columns) or RIP+ (red columns). All data are from at least 3 independent experiments in parallel cultures with error bars representing standard deviations.

FIG. 4. IGF-I instructs neural progenitor cells to commit to an oligodendroglial lineage and increases the proliferation of committed oligodendrocytes. (A) Adult neural progenitor cell states in culture: α, proliferation rate of progenitors (RIP−); β, differentiation rate of progenitors (RIP−) to oligodendrocytes (RIP+); δ, cell death rate for progenitors (RIP−); γ, proliferation rate for oligodendrocytes (RIP+); ε, cell death rate for oligodendrocytes (RIP+). (B-C) Comparison between direct measurements (columns) and modeling analyses (lines) of the total cell numbers (B) and of the percentage of RIP+ cells (C). Black columns are insulin-free (no IGF-I) conditions and blue columns are 500 ng/ml IGF-I conditions. No significant differences were found between the direct measurements and the values predicted by the model in each case. All data were normalized to values at the 12-h time point within each condition due to variability in cell behavior at time 0 (4 hours after plating). (D) The proliferation rates for RIP−(α) and RIP+ (γ) cells in insulin-free (no IGF-I) conditions (black line) and treated with 500 ng/ml IGF-I (blue and red lines). (E) The differentiation rate for RIP−(β) cells treated with 500 ng/ml IGF-I. These data are representative of at least 3 independent experiments. Error bars (except in D and E) represent standard deviations. The error bars in D and E represent the upper and lower bounds for proliferation (α, γ) and differentiation (β) rates.

FIG. 5. IGF-I-mediated oligodendrocyte differentiation involves an inhibition of BMP signaling. (A-B) Cells treated with 500 ng/ml IGF-I alone (A) or in combination with 50 ng/ml BMP2 (B). Cells are stained for an oligodendrocyte marker (RIP) and DAPI. (C) Quantification of RIP+ or cells treated with 500 ng/ml IGF-I alone or with different doses of BMP2 (0.05-50 ng/ml) in 4-day cultures. (D-E) Cells treated with 50 ng/ml IGF-I alone or in combination with 500 ng/ml Noggin. (F) Quantification of cells treated with different doses of IGF-I (20-1000 ng/ml) alone (blue columns) or in conjunction with 500 ng/ml Noggin (black columns). Significant differences are indicated with an asterisk (p<0.05, t-test). (G) Cells treated with a combination of IGF-I, BMP2 and Noggin. (H) Quantification of RIP+ cells in IGF-I alone, IGF-I+BMP2 and IGF-I+BMP2+Noggin. For G and H, concentrations are as follows: IGF-I (500 ng/ml), BMP2 (50 ng/ml), Noggin (500 ng/ml). Scale bar, 50 μM. All error bars represent standard deviations.

FIG. 6. IGF-I-induction of oligodendrocyte differentiation is associated with an upregulation of BMP antagonists, Noggin and Smad6, 7. (A-C) Q-PCR analyses and quantification of relative fold change (relative to GAPDH internal control) of Noggin (A) Smad6 (B) and Smad7 (C). RNA was harvested from cultures at time 0 (4 hours after plating) or from cultures treated with 500 ng/ml IGF-I after 12-, 24-, 36-, and 48-h time points. Significant differences are indicated with an asterisk (p<0.05, t-test) and all error bars represent standard deviations.

FIG. 7. IGF-I overexpression in the hilus promotes oligodendrocyte differentiation in vivo. (A-J) Representative images of brain sections focusing in on the hilar region in animals injected with rAAV-β-gal controls (A-D, I) and rAAV-IGF-I (E-H, J). Sections were triple-labeled with antibodies to oligodendrocyte markers RIP (A, E) and MBP (B, F), and an astrocyte marker GFAP (C, G). Merged images are shown in D and H; RIP is in red, MBP is in blue, GFAP is in green. (I-J) Representative sections stained with the oligodendrocyte marker GST-π in red and DAPI to visualize cell nuclei. White arrows indicate cells that are GST-π-positive. (K) The average number of cells in the hilus (in 3 adjacent fields distal to the injection site) per section in which GST-π was detected in each animal group (rAAV-β-gal animals, n=3; rAAV-IGF-I animals, n=4) is plotted. The asterisk indicates that values are significantly different between control and IGF-I overexpressed animals (p<0.001, t-test), and error bars represent standard deviations.

FIG. 8. Proposed model for the role of IGFs in multipotent neural progenitor cell fate specification: oligodendroglial and neuronal fate commitment at the expense of astroglial fates? (A) BMP signaling has been shown to stimulate astroglial differentiation and inhibit neuronal and oligodendroglial differentiation. (B) Activation of IGF-I receptor on multipotent neural progenitor cells by IGFs leads to the upregulation of Noggin and Smad6, 7. Since Noggin and Smad6, 7 inhibit BMP signaling, the net effects of IGF signaling are a block in astrocyte differentiation and an induction of neuronal and oligodendroglial differentiation. Alternatively, IGF-instructive effects on oligodendrocyte differentiation could occur in a Noggin/Smad6, 7-independent pathway.

### DETAILED DESCRIPTION OF THE INVENTION

Definitions

“Oligodendrocyte” refers to a central nervous system cell that has the ability or potential to produce myelin. Oligodendrocytes typically express one or more of the following markers: RIP, 4, NG2, myelin basic protein, proteolipid protein, myelin-associated glycoprotein, myelin/oligodendrocyte protein, galactocerebroside, and MOG. Morphological characteristics are as follows: The-cell soma ranges from 10 to 20 m and is roughly globular and more dense than that of an astrocyte. The margin of the cell is irregular and compressed against the adjacent neuropil. Few cell processes are seen, in contrast to the astrocyte. Within the cytoplasm, many organelles are found. Parallel cisternae of the rough ER and a widely dispersed Golgi apparatus are common. Free ribosomes occur, scattered amid occasional multivesicular bodies, mitochondria and coated vesicles. Distinguishing the oligodendrocyte from the astrocyte are the apparent absence of glial filaments and the constant presence of 24-nm microtubules. Microtubules are most common at the margins of the cell, in the occasional cell process and in the cytoplasmic loops around myelin sheaths. Lamellar dense bodies, typical of oligodendrocytes, are also present. The nucleus is usually ovoid, but slight lobation is not uncommon. The nucleochromatin stains heavily and contains clumps of denser heterochromatin; the whole structure is sometimes difficult to discern from the background cytoplasm. Desmosomes and gap junctions occur between interfascicular oligodendrocytes. Oligodendrocytes have been separated into three groups based on location, stainability and DNA turnover. Their three classes correspond to satellite, intermediate and interfascicular, or myelinating, oligodendrocytes. Satellite oligodendrocytes are small (˜10 m), restricted to gray matter and closely applied to the surface of neurons. They are assumed to play a role in the maintenance of the neuron and are potential myelinating cells. Interfascicular oligodendrocytes are large (˜20 m) during myelination but, in the adult, range from 10 to 15 m, with the nucleus occupying a large percentage of the cell volume. Intermediate oligodendrocytes are regarded as satellite or potential myelinating forms. The nucleus of these cells is small, the cytoplasm occupying the greater area of the soma.

“Multipotent neural stem or neural progenitor cell” refers to an multipotent cell, preferably adult-derived, in the neural cell lineage. A stem or progenitor cell is a cell which is capable of reproducing itself and also of ultimately differentiating into all the cell types in the neural cell lineage, including neurons and the glial cells astrocytes and oligodendrocytes. Thus, the neural stem cells are multipotent as they can differentiate into more than one neural cell type. Often the neural or progenitor stem cell is derived from the central nervous system, e.g., the hippocampus. The stems cells are preferably adult stem cells but can be embryonic stem cells. A stem or progenitor cell is one that is not committed to a particular differentiated cell type but can become one or more types of cells. In contrast, a committed progenitor cell has can only become one type of differentiated cell.

“Growth factor” refers to a serum or extracellular protein ligand that stimulates cell division when it binds to its cell-surface receptor.

“IGF-1” or “somatomedin C” refers to a growth factor polypeptide which (1) shares substantial sequence similarity with a native mammalian IGF-1, particularly the native human IGF-1; and (2) possesses a biological activity of the native mammalian IGF-1. The native human IGF-1 is a polypeptide of 70 amino acids with a molecular weight of 7648 daltons (see, for example, U.S. Pat. No. 4963,665 and U.S. Pat. No. 5,231,178). IGF-1 precursers (two variants 1-a and 1-B) are also known. A polypeptide which shares “substantial sequence similarity” with the native human IGF-1 is at least about 60% identical with a native mammalian, preferably native human, IGF-1 at the amino acid level. The IGF-1 is preferably at least about 70%, more preferably at least about 80%, yet more preferably at least about 90%, and most preferably at least about 95% identical with the native mammalian IGF-1 at the amino acid level. Thus, the term “IGF-1” encompasses IGF-1 analogs which are the deletional, insertional, or substitutional mutants of the native IGF-1. Furthermore, the term “IGF-1” encompasses the IGF-1s from other species and the naturally occurring variants thereof. Exemplary human IGF-1 sequences (precursor and processed form) are provided by the following accession numbers: 0912651A, CAA01954, AAA52538, and AAA52537, see also Rotwein et al., J. Biol. Chem. 261:4828-4832 (1986); Jansen et al., Nature 306:609-611 (1983); Le Bouc et al., FEBS Letts. 196:108-112 (1986); Steenbergh et al., Biochem. Biophys. Res. Commun. 175:507-514 (1991); and Sandberg-Nordqvist et al., Brain Res. Mol. Brain. Res. 12:275-277 (1992).

“Defined media” refers to a cell culture medium in which all the components are known. Preferably, the medium used in the present application is Gibco N2, serum free, minus insulin. IGF-1 is the only exogenously added or recombinantly produced growth factor (e.g., the added IGF-1 may be native or recombinant, or the cells may be engineered to produce recombinant IGF-1). The medium may also contain non-recombinant, native, endogenous growth factors produced by the cultured cells. For example, the medium used herein comprises only one added recombinant growth factor, IGF-1, and does not contain any other exogenously added growth factors such as FGF-2. However, the cultured cells may endogenously produce native factors such as FGF-2.

A “neurodegenerative disease or condition” is a disease or medical condition associated with neuron loss or dysfunction. Examples of neurodegenerative diseases or conditions include neurodegenerative diseases, brain injuries, spinal cord injuries, or CNS dysfunctions. Neurodegenerative diseases include, for example, de-myelination diseases, Alzheimer's disease, age-related dementia, multiple sclerosis (MS), macular degeneration, glaucoma, diabetic retinopathy, peripheral neuropathy, Huntington's disease, amyotrophic lateral sclerosis (ALS), and Parkinson's disease. Brain injuries include, for example, stroke (e.g., hemorrhagic stroke, focal ischemic stroke or global ischemic stroke) and traumatic brain injuries (e.g. injuries caused by a brain surgery or physical accidents). Spinal cord injuries include traumatic injuries caused by surgery or physical accidents. CNS dysfunctions include, for example, major depression, bipolar disorder, epilepsy, anxiety, neurosis, and psychotic disorders such as schizophrenia.

“Treating or ameliorating” means the reduction or complete removal of the symptoms of a disease or medical condition.

A mammal “suspected of having a neurodegenerative disease or condition” is a mammal which is not officially diagnosed of the neurodegenerative disease or condition but shows a symptom of the neurodegenerative disease or condition, is susceptible to the neurodegenerative disease or condition due to family history or genetic predisposition, or has had the neurodegenerative disease or condition before and is subject to the risk of recurrence.

“Transplanting” a composition into a mammal refers to introducing the composition into the body of the mammal by any method established in the art. The composition being introduced is the “transplant”, and the mammal is the “recipient”. The transplant and the recipient may be syngeneic, allogeneic or xenogeneic. Preferably, the transplantation is an autologous transplantation.

An “effective amount” is an amount of a therapeutic agent sufficient to achieve the intended purpose. For example, an effective amount of a growth hormone to increase the number of neural stem cells is an amount sufficient, in vivo or in vitro, as the case may be, to result in an increase in neural stem cell number. An effective amount of a growth hormone to treat or ameliorate a neurodegenerative disease or condition is an amount of the growth hormone sufficient to reduce or remove the symptoms of the neurodegenerative disease or condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

“Culturing” refers to growing cells ex vivo or in vitro. A “stable cell culture” refers to a culture of cells, typically oligodendrocytes obtained by differentiating mammalian multipotent neural stem cells by contacting the cells with recombinant IGF-1. The culture contains at least about 10% oligodendrocytes, preferably at least about 40% oligodendrocytes as identified by morphologic features or markers. The stable cell cultures may comprise one or more different cell types. The oligodendrocyte cultures typically divide for approximately one week in culture after stimulation of differentiation with IGF-1.

The term “antibody” refers to a polypeptide encoded by an immunoglobulin gene or functional fragments thereof that specifically binds and recognizes an antigen).

The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)₂ may be reduced under mild conditions to break the disulfide linkage in the hinge region, thereby converting the F(ab)′₂ dimer into an Fab′ monomer. The Fab′ monomer is essentially an Fab with part of the hinge region (see Fundamental Immunology (Paul, ed., 4th ed. 1999)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies or those synthesized de novo using recombinant DNA methodologies (e.g., single chain Fv).

The term “immunoassay” is an assay that uses an antibody to specifically bind an analyte. The immunoassay is characterized by the use of specific binding properties of a particular antibody to isolate, target, and/or quantify the analyte.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences,one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to reference nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2x SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

The phrase “selectively associates with” refers to the ability of a nucleic acid to “selectively hybridize” with another as defined above.

The phrase “functional effects” in the context of assays for testing compounds that modulate differentiation of adult mammalian CNS multipotent neural stem cells into oligodendrocytes includes, e.g., development of oligodendrocyte morphological characteristics, marker RNA and protein expression e.g., (RIP, 4, NG2, myelin basic protein, proteolipid protein, myelin-associated glycoprotein, myelin/oligodendrocyte protein, galactocerebroside, MOG, etc.), enzyme expression, cellular proliferation, myelin production etc. A functional effect therefore includes ligand binding activity, the ability of cells to proliferate, apoptosis, and enzyme activity. “Functional effects” include in vitro, in vivo, and ex vivo activities.

“Inhibitors”, “activators”, and “modulators” are used to refer to activating, inhibitory, or modulating molecules identified using in vitro and in vivo assays for oligodendrocyte differentiation. Inhibitors are compounds that, e.g., partially or totally block activity, decrease, prevent, delay activation, inactivate, desensitize, or down regulate oligodendrocyte differentiation, e.g., antagonists. “Activators” are compounds that increase, open, activate, facilitate, enhance activation, sensitize, agonize, or up regulate oligodendrocyte differentiation, e.g., agonists. Inhibitors, activators, or modulators naturally occurring and synthetic ligands, antagonists, agonists, antibodies, peptides, cyclic peptides, nucleic acids, antisense molecules, siRNA, ribozymes, small chemical molecules and the like.

Samples or assays of adult neural stem cells that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, siRNA, lipid, fatty acid, polynucleotide, oligonucleotide, etc., to be tested for the capacity to directly or indirectly modulation oligodendrocyte differentiation. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

“Biological sample” include sections of tissues such as biopsy and autopsy samples, and frozen sections taken for histologic purposes. Such samples include blood, sputum, brain tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

In vitro and ex vivo Cell Culture

The cells of the present invention are cultured according to methods known in the art. Adult neural stem cells are typically derived from the CNS, often from the hippocampal or subventricular region, but can be derived from any suitable region of the brain or other source. The adult neural stem cells of the invention are isolated and cultured according to methods known in the art (see, e.g., Gage, et al., Proc Natl Acad Sci U S A. 92:11879-83 (1995); Palmer, et al., Mol Cell Neurosci. 8:389-404 (1997); Ray, et al., Proc Natl Acad Sci USA. 90:3602-6 (1993)). Other methods of isolating neural stem cells are described, e.g., in U.S. patent publication 20030095956; U.S. patent publication 20030082160; U.S. patent publication 20020039724; U.S. Pat. Nos. 6,767,738, 6,498,018; 6,071,889, 5,980,885, 5,968,829, 5,851,832, and 5,750,376. Established neural stem cell lines can be used as well as primary cultures. The cells can also be genetically engineered to produce recombinant IGF-1 using methods known in the art, instead of exogenously adding IGF-1 to the cell culture medium. Inducible promoters can be used to activate IGF-1 expression conditionally using methods known to those of skill in the art.

Suitable cell culture methods and conditions can be determined by those of skill in the art using known methodology (see, e.g., Freshney et al., CULTURE OF ANIMAL CELLS (3rd ed. 1994)). In general, the cell culture environment includes consideration of such factors as the substrate for cell growth, cell density and cell contract, the gas phase, the medium, and temperature.

Typically plastic dishes or flasks are used. Other artificial substrates can be used such as glass and metals. The substrate is often treated by etching, or by coating with substances such as collagen, chondronectin, fibronectin, and laminin. The type of culture vessel depends on the culture conditions, e.g., multi-well plates, petri dishes, tissue culture tubes, flasks, and the like. Cells are grown at optimal densities that are determined empirically based on the cell type. For example, before adherence, a typical cell density for mononuclear cell cultures varies from about 1×10⁶ to about 1×10⁸ per ml of medium, and after adherence the typical cell density is about 1×10⁴ to about 1×10⁶ cells per ml.

Important constituents of the gas phase are oxygen and carbon dioxide. Typically, atmospheric oxygen tensions are used for the cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration is 5%.

Cultured cells are normally grown in an incubator that provides a suitable temperature, e.g., the body temperature of the animal from which is the cells were obtained, accounting for regional variations in temperature. Generally, 3720 C. is the preferred temperature for cell culture. Most incubators are humidified to approximately atmospheric conditions.

Defined cell media are available as packaged, premixed powders or presterilized solutions. Examples of commonly used media include Iscove's media, AIM-V, RPMI 1640, DMEM, and McCoy's Medium (see, e.g., GibcoBRL/Life Technologies Catalogue and Reference Guide; Sigma Catalogue). Defined cell culture media are often supplemented with 5-20% serum, e.g., human horse, calf, and fetal bovine serum. Preferably the serum is 10% non-heat inactivated human serum (Sigma). The culture medium is usually buffered to maintain the cells at a pH preferably from 7.2-7.4. Other supplements to the media include, e.g., antibiotics, amino acids, sugars, and growth factors.

Methods of Drug Screening

One embodiment of the invention provides methods of screening to identify compounds that inhibit or activate the differentiation of mammalian multipotent neural stem cells into oligodendrocytes. A culture of neural stem cells is cultured with IGF-1 described herein and is also contacted with a candidate compound. The effect of the compound on oligodendrocyte differentiation is determined by detecting oligodendrocytes (by morphology or markers) using the methods described herein. A compound that decreases the differentiation relative to the levels of a cell culture that has not been contacted with the compound is identified as an inhibitor of differentiation. Typically an inhibitor decreases the level of differentiation by about 10%, 20%, 30%, 40%, 50%, 60%, 70%, 80%, 90% or more relative to the level of differentiation in the absence of the compound. Similarly, a compound that increases the differentiation relative to the levels of a cell culture that has not been contacted with the compound is identified as an activator of differentiation. Typically an activator increases the level of differentiation by about 10%, 20%, 30%, 40%, 50%, 60%, 70%, 80%, 90% or more relative to the level of differentiation in the absence of the compound.

Suitable candidate compounds include, for example, proteins, peptides, anti-sense oligonucleotides, siRNA, ribozymes, antibodies, and small organic molecules.

In some embodiments, variants of a chemical compound (i.e., a “lead compound”) that modulates oligodendrocyte differentiation production are created and evaluated for their ability to modulate. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify compounds that modulate oligodendrocyte differentiation are amenable to high throughput screening. High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipeting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems.

In vivo and ex vivo Methods of Treatment of Disease States

The growth hormone, IGF-1, can be provided directly to a subject by any method established in the art, for the treatment of a neurodegenerative condition as described herein. For example, IGF-1 can be administered intravascularly, intrathecally, intravenously, intramuscularly, subcutaneously, intraperitoneally, topically, orally, rectally, vaginally, nasally, by inhalation or directly into the brain via a cannula or other known technique. When the factor is not directly delivered into the brain, a blood brain barrier permeabilizer can be optionally included to facilitate entry into the brain. Blood brain barrier permeabilizers are known in the art and include, by way of example, bradykinin and the bradykinin agonists described in U.S. Pat. Nos. 5,686,416; 5,506,206 and 5,268,164 (such as NH.sub.2-arginine-proline-hydroxyproxyproline-glycine-thienylala-nine-serine-proline-4-Me-tyrosine.psi.(CH.sub.2NH)-arginine-COOH). Alternatively, the factors can be conjugated to the transferrin receptor antibodies as described in U.S. Pat. Nos. 6,329,508; 6,015,555; 5,833,988 or 5,527,527. The factors can also be delivered as a fusion protein comprising the factor and a ligand that is reactive with a brain capillary endothelial cell receptor, such as the transferrin receptor (see, e.g., U.S. Pat. No. 5,977,307). For ex vivo administration, cells are administered to the brain according to methods known to those of skill in the art and using doseages according to those of skill in the art. The cells can be differentiated oligodendrocytes or cells engineered to express recombinant IGF-1 and thereby differentiate in vivo.

### EXAMPLES

The following examples are offered to illustrate, but not to limit the claimed invention.

### Example 1

### IGF-I-Induced Oligodendrocyte Differentiation

Results

IGF-I-Induced Oligodendrocyte Differentiation

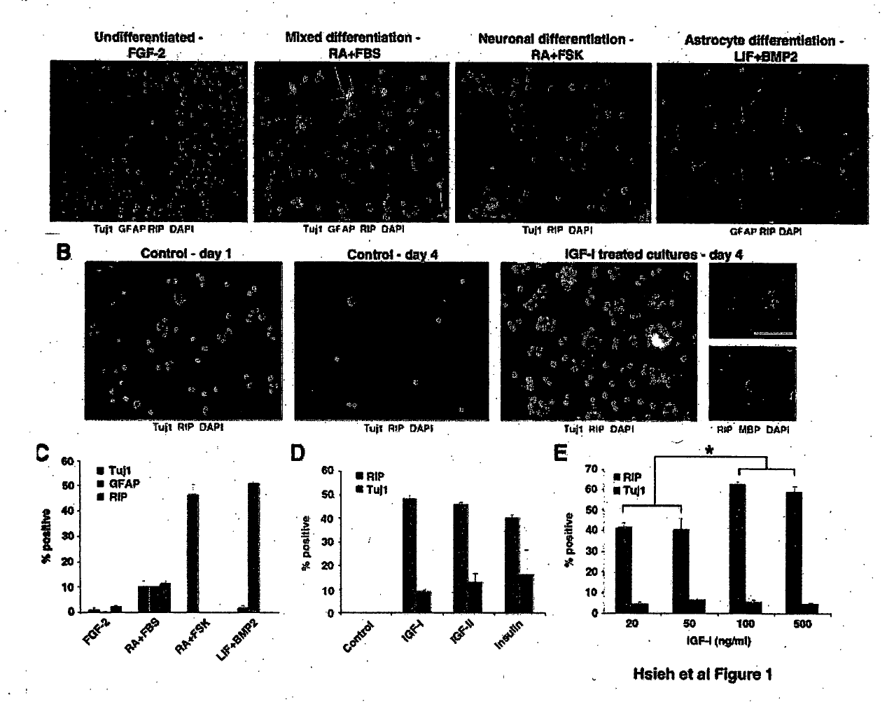

We first tested the action of exogenous IGF-I on the differentiation of hippocampus-derived adult neural progenitor cells. These neural progenitor cells have stem cell properties in vitro: (1) they self-renew in the presence of basic fibroblast growth factor (FGF-2), (2) single genetically marked clones can differentiate into all three main CNS cell types in vitro (neurons, oligodendrocytes, and astrocytes) and when grafted back to adult hippocampus in vivo, and (3) they express progenitor cell markers such as nestin, but lack markers of lineage-specific differentiation (FIG. 1A) (Gage et al., 1995; Palmer et al., 1997). To confirm that the adult neural progenitor cell population in our model system is indeed multipotent, we first used various standard differentiation paradigms and evaluated the expression of lineage markers. Differentiation with retinoic acid (RA, 1 μM) and fetal bovine serum (FBS, 0.5%) resulted in mixed numbers of Tuj1+ neurons, GFAP+ astrocytes, and RIP+ oligodendrocytes (FIG. 1A). Neuron-enriched differentiation can be achieved with RA (1 μM) and forskolin (FSK, 5 μM) (personal communication, Vi Chu) and astrocyte-enriched differentiation with leukemia inhibitory factor (LIF, 50 ng/ml) and bone morphogenetic protein-2 (BMP2, 50 ng/ml) (Nakashima et al., 1999) (FIG. 1A). The quantification of lineage-specific differentiation is shown (FIG. 1C). These results are in agreement with previous reports (Palmer et al., 1997) and indicate that the majority of the adult neural progenitor cells within the bulk population are multipotent progenitors, and not restricted neuronal or glial progenitors.

To determine the effects of IGF-I on neural progenitor cell differentiation, we compared cells treated with and without IGF-I over a 4-day period. All experiments were performed in insulin-free N2 medium to avoid its actions on insulin and/or IGF-I receptors. When FGF-2 was withdrawn from neural progenitor cells and the cells were cultured in the absence of insulin for 4 days, lineage-specific differentiation was not seen (FIG. 1B). There was also a dramatic decrease in cell survival, consistent with the important effects of IGFs for preventing cell death (Vincent and Feldman, 2002). We found that neural progenitor cells cultured in the presence of 500 ng/ml IGF-I differentiated into a large percentage of RIP+ cells (approximately 45-55%) (FIG. 1, B and D). RIP is a marker for both immature and mature oligodendrocytes, in that it stains pre-ensheathing oligodendrocytes through myelinating stages and associated myelin (Friedman et al., 1989). In addition to RIP staining, these cells exhibited small and round somata with the characteristic web-like oligodendrocyte morphology within 4 days (FIG. 1B). Approximately 23% of the total cells were also positive for myelin basic protein (MBP), a late-appearing marker for myelinating oligodendrocytes (Akiyama et al., 2002), at the 4-day time point (FIG. 1B).

Furthermore, 500 ng/ml of IGF-II or insulin could also promote the preferential differentiation of neural progenitor cells into oligodendrocytes (FIG. 1D). The IGF-mediated oligodendrocyte differentiation is likely to occur through an activation of IGF-I receptors; addition of IGF-I, IGF-II, or insulin at different concentrations (20-1000 ng/ml) showed a rank order of potency IGF-I>IGF-II>insulin, which is consistent with the pharmacology of the IGF-I receptor (LeRoith et al., 1993). RT-PCR analysis showed IGF-I receptor mRNA expression in neural progenitors (unpublished data). There was also a small percentage of neural progenitor cells that differentiated into Tuj1+ neurons (approximately 5-15%) with IGF-I or IGF-II or insulin treatment (each at 500 ng/ml), with few to no GFAP+ astrocytes (FIG. 1D). The IGF-I-induced oligodendrocyte differentiation occurred in a dose-dependent manner, whereas the percentage of Tuj1-positive cells did not appear to change at any of the tested concentrations (FIG. 1E).

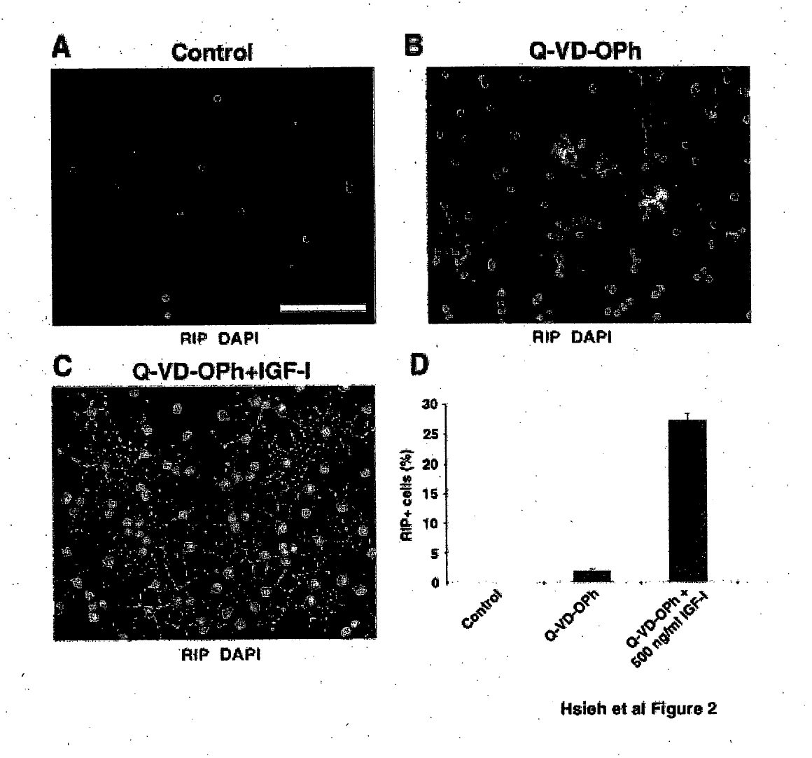

The severe and rapid death of neural progenitor cells cultured in the absence of IGF-I left open the possibility that FGF-2 withdrawal alone could be promoting oligodendrocyte differentiation and that the addition of IGF-I merely promoted oligodendrocyte survival. To address this, we kept neural progenitor cells alive with the broad caspase inhibitor Q-VD-OPh (Caserta et al., 2003) and assessed neural progenitor differentiation with and without IGF-I in 2-day cultures (FIG. 2, A-D). In the absence of Q-VD-OPh in insulin-free N2 medium, there was decreased cell survival evidenced by the presence of fragmented DAPI-stained nuclei and a general disappearance of cells (FIG. 2A). Addition of 2 □M Q-VD-OPh to the cells promoted the majority of the cells to survive as seen by smooth DAPI-stained nuclei and a general persistence of cells, without obvious effects on proliferation or differentiation (FIG. 2B). There was a small amount of RIP+ differentiation (approximately 2%) in Q-VD-OPh-treated cultures, presumably due to spontaneous differentiation upon FGF-2 withdrawal. The absolute number of cells does not significantly change in Q-VD-OPh-treated cultures compared to cells just after plating (unpublished data). Only when neural progenitors were cultured with 500 ng/ml IGF-1, we observed a large increase in RIP+ cells in 2-day cultures (FIG. 2C). The quantification of oligodendrocyte differentiation is shown (FIG. 2D). These data suggest that IGF-1 can directly induce multipotent neural progenitor cells to differentiate into oligodendrocytes, instead of merely promoting the survival of differentiated oligodendrocytes.

The oligodendrocyte-promoting effects of IGF-I were confirmed with an independently derived rat hippocampus progenitor cell line, as well as a clonally derived line (unpublished data). Furthermore, primary cultures of multipotent neural progenitor cells derived from the whole brain of P10 ICR mice also exhibited a preferential differentiation into oligodendrocytes upon IGF-I treatment (unpublished data). These data suggest that IGFs (IGF-I, -II) and insulin, through an activation of IGF-I receptors, are important in stimulating the differentiation and maturation of multipotent adult neural progenitor cells into oligodendrocytes.

IGF-I-induced increase in oligodendrocytes is attributable to an instructive differentiation, and a subsequent proliferation of committed oligodendrocytes

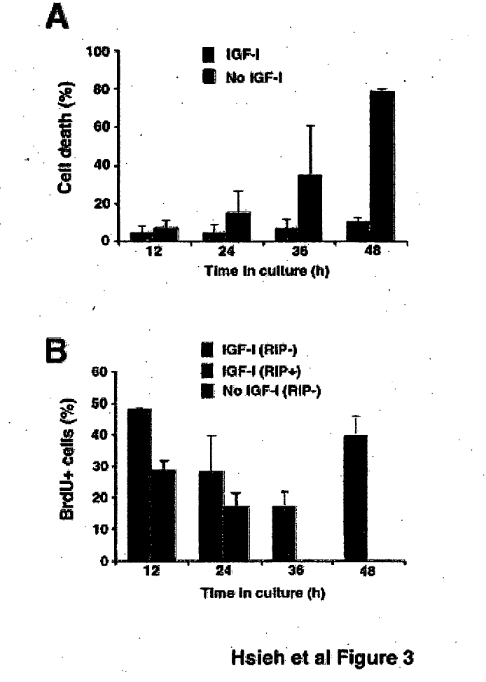

Although these results establish IGF-I as an inducer of oligodendrocyte differentiation for a population of multipotent neural progenitor cells, it is more complicated to quantitatively assess the instructive vs selective effects of IGF-I. We therefore determined whether the increase of oligodendrocytes after IGF-I treatment could be due to a combination of selective oligodendrocyte survival, increased proliferation of progenitors, and/or instructive differentiation of progenitor cells to an oligodendroglial lineage. We first directly measured the effect of IGF-I on cell survival. We asked whether a selective decrease in the death of oligodendrocytes (and/or oligodendrocyte progenitors) could explain the net increase in number of oligodendrocytes (defined by the expression of RIP, a marker of both immature and mature oligodendrocytes). If IGF-I had a selective survival effect on oligodendrocytes and/or their progenitors, we would expect to see a change in cell death due to the increased death of other cell types. To quantify cell death of the progeny of neural progenitor cells, living cultures were stained with propidium iodide (1 μg/ml), which stains dead cells, and Hoechst 33342 (1 μg/ml), which stains live and dead cells. Staining of live, rather than fixed, cultures was used to avoid underestimating cell death due to possible detachment of dying and/or dead cells from culture substrates. This assay revealed a relatively small percentage of dead/dying cells, and one-way ANOVA analysis revealed no significant change (p=0.3930) in the percentage of cells that died at any of the time points (FIG. 3A). The amount of cell death in IGF-I-treated cultures was similar to the amount of cell death under standard proliferating conditions with FGF-2 (unpublished data). Cultures grown in the absence of IGF-I exhibited a progressive increase in overall cell death, reinforcing the role of IGF-I as an important factor for cell survival. Since there was minimal death and no significant difference in the percent of dead/dying cells at each of the time points in the IGF-I-treated cultures, a selective survival of oligodendrocyte progenitors or oligodendrocytes does not appear to have a significant role in the increased net oligodendrocyte differentiation with IGF-I treatment.

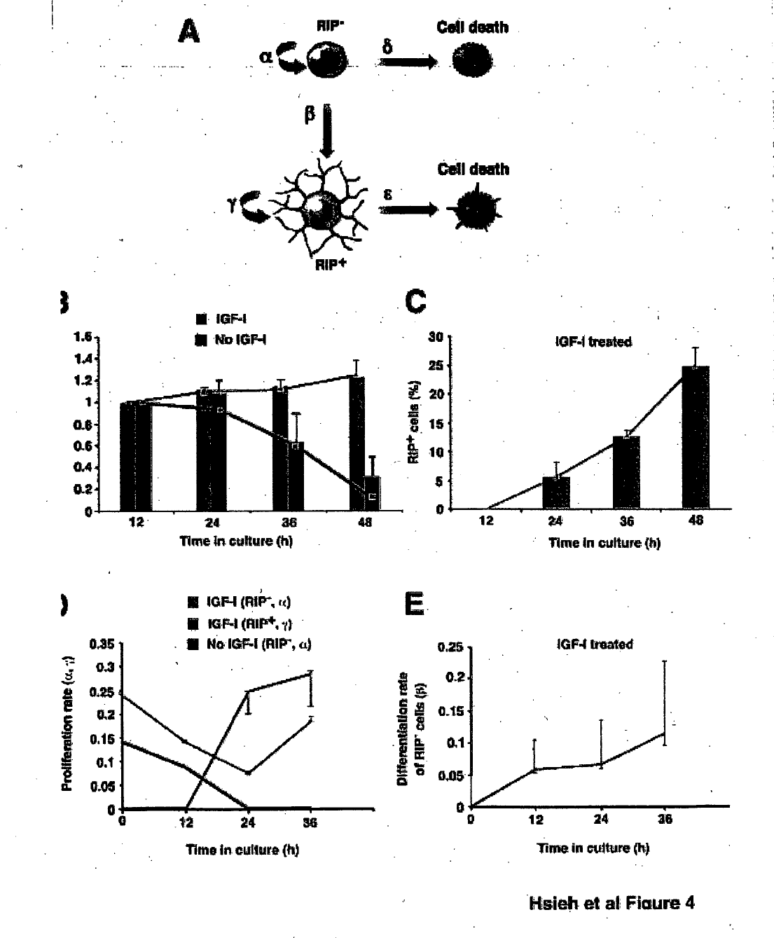

We next determined whether proliferation of progenitors might contribute to the observed increase in oligodendrocyte differentiation. Bromodeoxyuridine (BrdU) (2.5 μM) was added to parallel cultures for 12 hours at times 0-, 12-, 24-, and 36-h after plating, followed by fixation and quantification of total cell numbers and BrdU+ cells. We counted the progenitor cells (evidenced by a lack of RIP staining) that were labeled with BrdU at each time point. For cells grown in the absence of IGF-I, we observed a progressive decrease in the percentage of cells that were proliferating (BrdU+) during the 48-h experiments (FIG. 3B). There was a marked decrease in proliferation during the 36- and 48-h periods, probably due to limited cell survival in the absence of IGF-I. In cultures treated with IGF-I, the percentage of RIP− cells that incorporated BrdU also exhibited a decreasing trend in the first three time periods, but to a lesser extent compared to the cells grown without IGF-I (FIG. 3B, blue columns). This initial decrease in proliferation is most likely due to FGF-2 mitogen withdrawal. The fact that we could already observe the generation of RIP+ oligodendrocytes at the 24-h time point (FIG. 4C), coupled with the observation that there was a decrease in BrdU uptake of RIP− cells, suggests that IGF-I did not have a major proliferative effect on progenitor cells.

These data taken alone may not be sufficient to fully evaluate the effects of IGF-I on proliferation of RIP− progenitors, because if there is also a differentiation effect (conversion of RIP− to RIP+ cells), a substantial decrease in the percentage of RIP− cells that were BrdU+ may be seen. Therefore, to separate the IGF-I effects on proliferation of progenitors from the instructive differentiation of progenitors to oligodendrocytes, we developed a mathematical model (as shown in FIG. 4A). This modeling approach has previously been used to quantify the effects of hippocampal astrocytes on the proliferation and neuronal differentiation of adult neural progenitor cells (Song et al., 2002). To distinguish the proliferation and differentiation rates, the model required that we count the number of RIP+ cells that were either positive or negative for BrdU at each time point. There was a progressive increase in the overall number of RIP+ cells over time in the presence of IGF-I (FIG. 4C). We also stained the cultures at each time point for Tuj1+ neurons and GFAP+ astrocytes to determine if the model should account for the differentiation to alternative cell types; since neuronal differentiation was minimal and astrocyte differentiation was undetectable, these possibilities did not merit inclusion into the model. Interestingly, after 24 hours, there was also a significant increase in RIP+ cells positive for BrdU, suggesting that committed oligodendrocytes may still be proliferating in the presence of IGF-I (FIG. 3B, red columns). Because of this finding, we decided to separate the proliferation rate of RIP− (defined as α) and RIP+ cells (γ), as well as the differentiation rate (β) of RIP− to RIP+ cells. The overall death rate measured by Hoechst dye in live cultures could not be experimentally separated into a RIP− death rate (δ) and a RIP+ death rate (ε) (Song et al., 2002). Therefore, the model was bounded by the two possibilities that all cell death is from only RIP− or only RIP+ cells. We also considered the more probable assumption that the death rates for both RIP− and RIP+ cells were equal.

Six equations were used to derive values for these rates. The first equation describes the differential gain in RIP− cells:
dN^{RIP-}/dt=(α−δ)N^{RIP-}−βN^{RIP-} (b 1)
where N^{RIP-}is the number of RIP− cells, α is the proliferation rate of RIP− cells (per 12-h interval), β is the differentiation rate of RIP− into RIP+ cells, and δ is the death rate of RIP− cells. Similarly for RIP+ cells,
dN^{RIP+}/dt=(γ−ε)N^{RIP+}+βN^{RIP-} (2)
where N^{RIP+} is the number of RIP+ cells, δ is a proliferation rate of RIP+ cells, and ε is the RIP+ death rate. The sum of (1) and (2) is the equation defining total cell count:
dN^{Total}/dt=(α−δ)N^{RIP-}+(γ−ε)N^{RIP+} (3)

The time-course experiments were performed in 12-h intervals, allowing for measurement of the proliferation and differentiation rates at each 12-h time interval—αᵢ is the proliferation rate of RIP− cells at time ‘i’ and so forth. BrdU uptake experiments allow for direct measurements of the number of new cells that were generated within the time interval:
# BrdUᵢ₊₁=2γᵢN^{RIP+}ᵢ+2αᵢN^{RIP-}_{I} (4)
where #BrdUᵢ₊₁ is the number of cells that incorporated BrdU between time ‘i’ and ‘i+1’. Inspection of BrdU+ cells that are negative or positive for RIP gives the equations:
# BrdUᵢ₊₁^{RIP-}=2αᵢN^{RIP-}ᵢ−βᵢ[βᵢ/(1+αᵢ)](1+αᵢ)]N^{RIP-}ᵢ (5),
# BrdUᵢ₊₁^{RIP+}=2γᵢN^{RIP+}ᵢ+βᵢ[αᵢ/(1+αᵢ)](1+αᵢ)]N^{RIP-}ᵢ (6)
where the βᵢ[αᵢ/(1+αᵢ)]N^{RIP-}ᵢ term represents the number of RIP− cells that may have divided then differentiated within the same 12-h interval [probability of differentiation βᵢ times the 2αᵢ/(1+αᵢ) term for probability that the differentiating cell is BrdU+, with a ½ correction for the order of events]. Since dead cells cannot be accurately separated into RIP+ and RIP− cells, the model is fitted under three possible assumptions: all death is RIP+ (δ=0); all death is RIP− (ε=0); and the death rates are equal (δ=ε). The proliferation equations ‘5’ and ‘6’ were solved with the differentiation equation ‘2’ to find values for αᵢ, βᵢ, and γᵢ.

To ensure that the model for oligodendrocyte differentiation accurately represented the biological system, we compared the values predicted by the model for total cell counts (equation 3) and cell differentiation (equation 2) to direct measurements at the time points between 12 hours and 2 days. No significant differences were found between direct measurements (columns) and predicted values (lines; FIG. 4, B and C). The model-derived values indicated that there was no increase in the proliferation rate of RIP− cells (α) over time, reinforcing the idea that the increased proliferation rate of oligodendrocyte progenitors was not the cause for the net increase in oligodendrocyte numbers (FIG. 4D, blue line). The model does predict an increase in differentiation rate (β) (FIG. 4E, blue line); together with the lack of change in the proliferation rate (α), this can only be taken to mean an increase in instructive differentiation of RIP− to RIP+ cells by IGF-I.

The increase in instructive differentiation by itself is not enough to account for the conversion of RIP− to RIP+ cells. The net increase in RIP+ cells must also include in the explanation an increase in the proliferation rate (γ) of RIP+ cells (FIG. 4D, red line). If the γ term was set to zero (no RIP+ proliferation), the model predicted significantly fewer RIP+ oligodendrocytes than were observed experimentally. Therefore, the only way to resolve the net increase in oligodendrocyte numbers according to the model is an instructive differentiation from adult multipotent neural progenitors to oligodendrocytes and a subsequent proliferation of committed oligodendrocytes in the presence of IGF-I.

IGF-I-Induced Oligodendrocyte Differentiation is Mediated Through an Inhibition of BMP Signaling

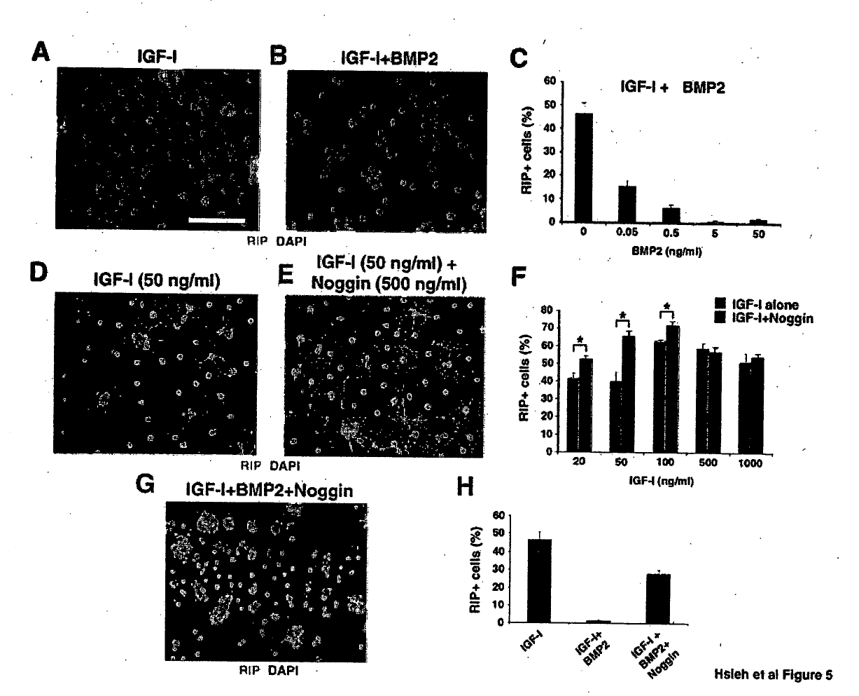

In the developing vertebrate telencephalon and spinal cord, BMPs have been shown to act as inhibitory signals for oligodendrocyte fate specification (Gross et al., 1996; Mekki-Dauriac et al., 2002). Additional studies have shown that oligodendrocyte lineage progression requires an active inhibition of BMP signaling (Mabie et al., 1999; Mehler et al., 2000). To gain insight into the molecular mechanism of IGF-I-induced oligodendrocyte differentiation, we asked whether IGF-I effects are mediated through an inhibition of BMP signaling. We first tested whether BMPs could inhibit adult neural progenitor cell oligodendrocyte differentiation. Addition of BMP2 in the presence of 500 ng/ml IGF-I resulted in a suppression of oligodendrocyte differentiation, as evidenced by a reduction in RIP+ cells compared with cultures treated with IGF-I alone (FIG. 5, A-C). Similar results were observed with BMP4 (unpublished data). This inhibition of oligodendrocyte differentiation is dose-dependent; higher concentrations of BMP2 (5-50 ng/ml) resulted in a greater suppression of oligodendrocyte differentiation, whereas partial differentiation could still be observed at lower BMP2 concentrations (0.05-0.5 ng/ml; FIG. 5C). These results are consistent with the ability of BMPs to repress oligodendrocyte differentiation.

BMP signaling can be inhibited by extracellular proteins, such as Noggin, that bind BMP ligands in a competitive manner, interfering with the ability of ligand binding to cognate cell-surface receptors (Balemans and Van Hul, 2002). If the IGF-I induction of oligodendrocyte differentiation is mediated through an inhibition of BMP signaling, the function of BMP antagonists could be involved. To test the involvement of BMP antagonists, we treated neural progenitor cells with different concentrations of IGF-I in conjunction with exogenous Noggin and compared the extent of oligodendrocyte differentiation to cultures treated with IGF-I alone (FIG. 5, D-F). When Noggin (500 ng/ml) was added to cultures treated with IGF-I (20-100 ng/ml), there was a significant increase in the percentage of oligodendrocytes compared to cultures treated with IGF-I alone (p<0.05, t-test) (FIG. 5F). Addition of Noggin (500 ng/ml) together with highest concentrations of IGF-I (500-1000 ng/ml) did not result in a significant change in the percentage of RIP+ oligodendrocytes compared with IGF-I alone (FIG. 5F), presumably due to saturating effects of IGF-I. We next wanted to rule out if Noggin by itself had any effects on oligodendrocyte differentiation and/or enhanced survival. Neural progenitor cells treated with 500 ng/ml Noggin alone in insulin-free media did not display any enhanced differentiation or survival effects. Finally, addition of Noggin (500 ng/ml) together with BMP2 (50 ng/ml) and IGF-I (500 ng/ml) efficiently reversed the inhibitory effects of BMP signaling on oligodendrocyte differentiation (FIG. 5, G and H).

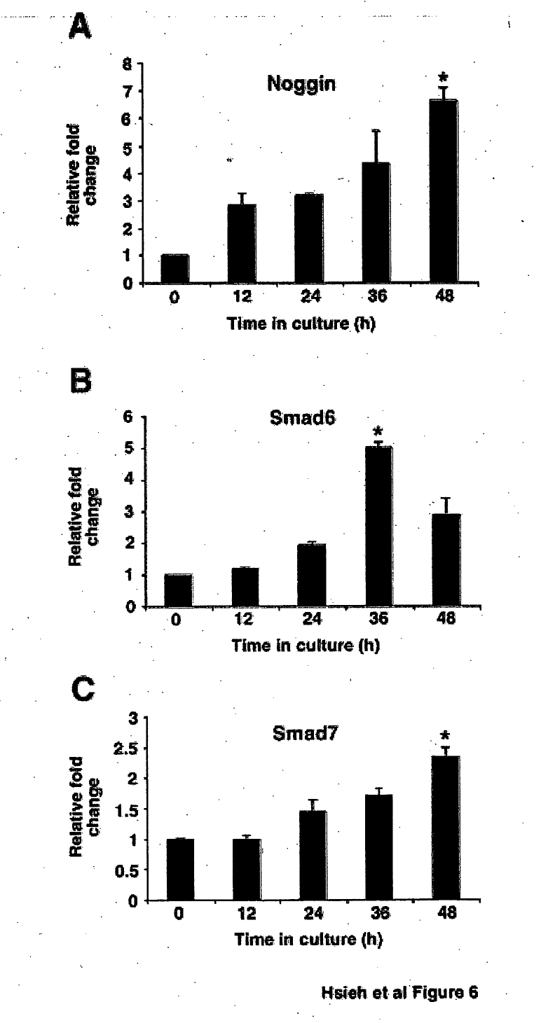

If IGF-I induction of oligodendrocyte differentiation involves the inhibition of BMP signaling, a change in the expression of BMP antagonists after IGF-I treatment might be observed. In addition to extracellular antagonists of BMP signaling, such as Noggin, intracellular proteins that can interfere with downstream BMP receptor signaling, called inhibitory Smads (Smad6, 7), have also been identified (Christian and Nakayama, 1999). Therefore, we performed Q-PCR analyses of cultures treated with IGF-I after various time points (up to 48 hours) and examined the relative fold change (normalized to an internal control; see Methods) of Noggin, Smad6, 7 (FIG. 6, A-C). Expression of Noggin, Smad6, 7 increased by at least 6-, 5-, and 2-fold, respectively, after IGF-I treatment (p<0.05, t-test). There may be an unexplained downregulation of Smad6 at the 48-h time point. In each case, an upregulation of gene expression is clearly observed with IGF-I induction of oligodendrocyte differentiation; however, the kinetics of upregulation suggest that IGF-I may not be directly upstream of Noggin and inhibitory Smads. Taken together, these results suggest that IGF-I instructive effects on oligodendrocyte differentiation are mediated, at least in part, through an inhibition of BMP signaling.

Effects of IGF-I on Oligodendrocytes in vivo

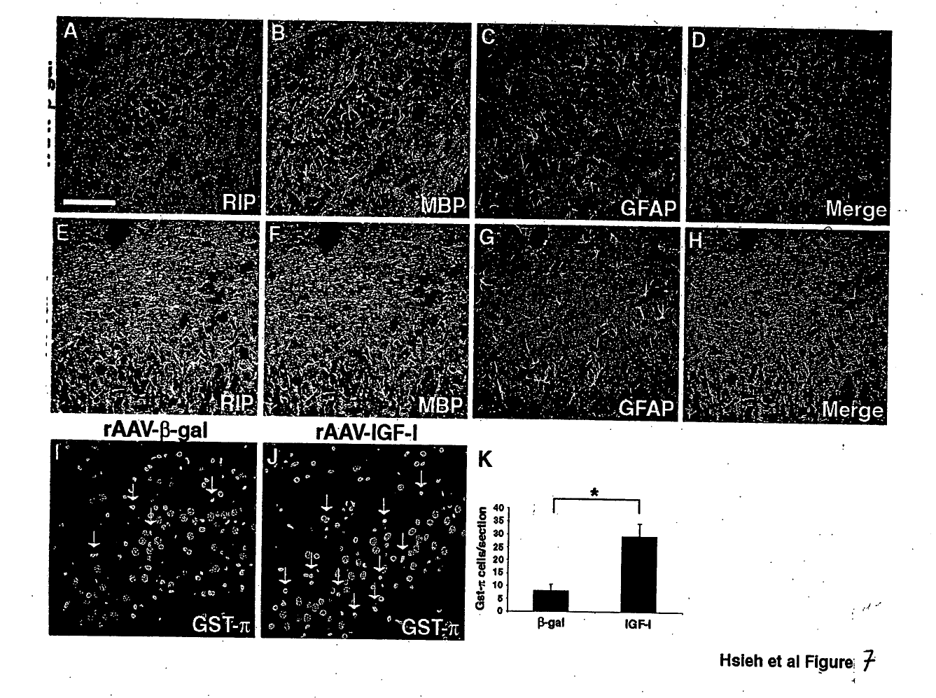

Our findings raised the question whether IGF-I also has effects on oligodendrocyte differentiation in vivo in the region where adult hippocampal neural progenitor cells normally reside. We therefore used adeno-associated virus (AAV) to overexpress IGF-I in the adult rat hippocampus. To evaluate effects on the endogenous oligodendrocyte population due to IGF-I overexpression in vivo, we used the oligodendrocyte marker RIP. We focused our analyses on the hilar region of the dentate gyrus, since there is a higher concentration of oligodendrocytes in this region. An increase of RIP staining in the hilus of rAAV-IGF-I-infected animals compared with rAAV-β-gal-infected controls was apparent (FIG. 7, A and E). Quantification of RIP immunofluorescence in the hilus of rAAV-β-gal-infected control animals resulted in an average pixel intensity of 45.49±2.01 (n=3 rats) compared to an average pixel intensity of 99.95±18.27 (n=4 rats) in rAAV-IGF-I-infected animals (p<0.05, t-test). Similar results were seen in at least three adjacent fields of the same section.

A similar trend was observed using MBP immunofluorescence as a marker for mature oligodendrocytes. The extent of MBP staining appeared more intense in the hilus of IGF-I-infected animals compared to control animals (FIG. 7, B and F). The average pixel intensity of MBP immunofluorescence in controls was 30.8±6.84 (n=3 rats) compared to an average pixel intensity of 53.53±5.72 (n=4 rats) in rAAV-IGF-I-infected animals (p<0.05, t-test). There was no observable difference in GFAP staining between control and IGF-I-infected animals, suggesting that there is not a general increase in all glial cell lineages in animals overexpressing IGF-I (FIG. 7, C and G).

Since we observed an increase in overall staining for various oligodendrocyte markers (RIP and MBP) after IGF-I overexpression in the adult hippocampus, we next determined if there was also a change in the number of oligodendrocytes. For quantification of oligodendrocytes, sections were labeled for the 7 isoform of glutathione S-transferase (GST-π). GST-π has been determined in many studies to label both immature and mature myelinating oligodendrocytes (Mason et al., 2001; Tansey and Cammer, 1991) and facilitates cell counting due to its predominant localization in oligodendrocyte cell bodies as well as in a few of the processes. The average number of GST-π-positive cells per section was 8.08±2.43 (n=3 rats) for rAAV-β-gal control animals and 29.14±5.04 (n=4 rats) for rAAV-IGF-I animals, which is a 3-fold difference (p<0.001, t-test) (FIG. 7, I-K). These results support that IGF-I is a regulator of oligodendrocyte differentiation in vivo as well as in vitro and ex vivo.

Discussion

We find that IGF-I, IGF-II and insulin could all independently stimulate the differentiation of adult multipotent neural progenitor cells into oligodendrocytes. Modeling analysis reveals that IGF-I provides an instructive regulation in oligodendroglial fate choice rather than a selective regulation in proliferation or survival of oligodendrocyte progenitors. IGF-I-treated cultures also demonstrate an increase in the proliferation of committed oligodendrocytes. Moreover, the finding that IGF-I overexpression in the adult hippocampus leads to an increase in oligodendrocyte markers supports IGF-I effects in vitro. This is the first example of a single factor that can induce the robust differentiation of multipotent neural progenitor cells into oligodendrocytes.

To address whether IGF-I effects are instructive and/or selective in nature, we use both experimental and modeling approaches. We first addressed the question whether IGF-I might have selective effects on cell survival. Since the frequency of cell death is low in IGF-I-treated cultures and there is no significant difference in the percentage of cells that are dying/dead at each of the time points analyzed, it is unlikely that the net increase in oligodendrocytes by IGF-I comes from the selective survival of committed oligodendrocyte progenitors that then go on to differentiate. Although there is massive cell death in the absence of IGF-I, this cell death applies widely across all types of neural progenitor cells. Treatment of cells with the broad caspase inhibitor, Q-VD-OPh, further reinforced the finding that IGF-I is not merely acting on cell survival. The addition of Q-VD-OPh is enough for the cells to survive, and not proliferate or differentiate in short-term cultures; only upon addition of IGF-I is there a massive increase in oligodendrocyte differentiation.

Next we addressed the question of selective effects on cell proliferation by IGF-I. The BrdU studies indicate that there is not an increase in proliferation at time points where oligodendrocyte differentiation is already occurring, suggesting that a selective proliferation of progenitors does not appear to have a major contribution to the net oligodendrocyte increase. To address the possibility that a robust differentiation effect by IGF-I might mask a proliferation effect, a model was used to separate the independent contributions of IGF-I on proliferation and differentiation. The model-derived data showed an increase in the rate of oligodendrocyte differentiation and not in the proliferation rate of RIP− progenitors. The model also showed an increase in the proliferation rate of committed oligodendrocytes in IGF-I-treated cultures. The simplest interpretation of our data, therefore, is that IGF-I acts to control the fate choice of multipotent adult neural progenitor cells to an oligodendroglial lineage, and that the net increase in oligodendrocytes by IGF-I is due to an instructive differentiation and an additional proliferation of committed oligodendrocytes.

Recently it has been reported that IGF-I can stimulate neurogenesis in the dentate gyrus (Aberg et al., 2000), as well as increase the proliferation and neuronal differentiation of EGF-responsive multipotent neural stem cells derived from E14 mouse striatum (Arsenijevic and Weiss, 1998; Arsenijevic et al., 2001). Although, it is not clear whether oligodendrocyte differentiation was examined in these studies, the possibility still remains that IGF-I may promote the differentiation of multipotent neural progenitor cells to both neuronal and oligodendrocyte lineages. In fact, our studies show that, in IGF-I-treated cultures, although the majority of the cells differentiate into oligodendrocytes, a small number of cells can differentiate into neurons. Since small numbers of cells differentiate into neurons, it was difficult to determine if the effects of IGF-I on neuronal differentiation were instructive or selective in nature, or whether a population of lineage-restricted neuronal progenitors exists that could survive and differentiate in the presence of IGF-I. Since we did not observe an increase in astrocyte differentiation with IGF-I treatment in vitro, or any apparent effects on the existing astrocyte population in vivo, it would appear that IGF-I effects on neural progenitor cells are restricted, at least in part, to oligodendrocyte and neuronal lineages. The possibility still remains that IGF-I could have instructive effects on astroglial fate commitment from a multipotent progenitor cell population in vivo.

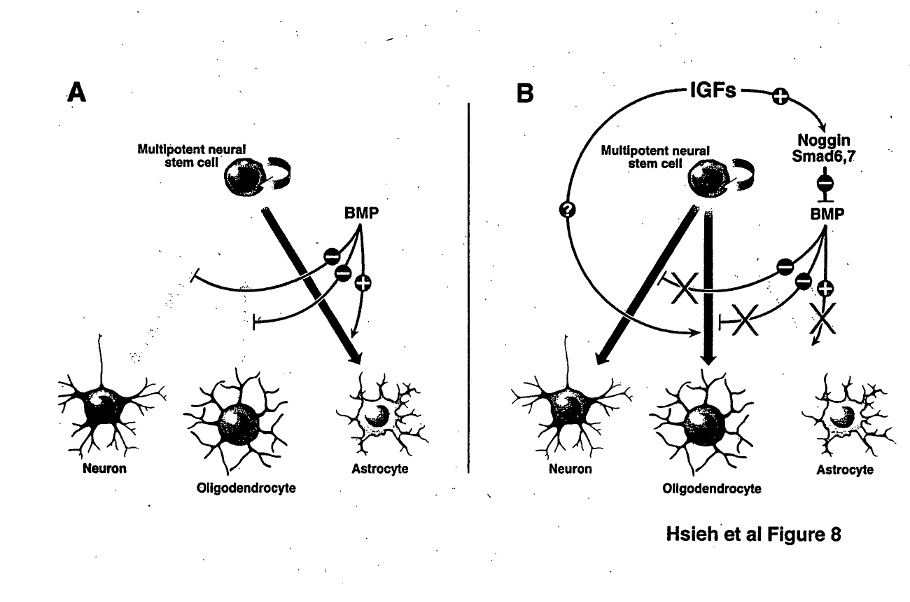

We hypothesized that oligodendrocyte differentiation of multipotent adult neural progenitor cells might utilize similar mechanisms as oligodendrocyte progenitors derived from the embryonic brain and spinal cord, and that IGF-I-induction of oligodendrocyte differentiation may involve an inhibition of BMP signaling. Our data suggest that BMPs can repress IGF-I-induced oligodendrocyte differentiation from adult neural progenitor cells. Furthermore, addition of Noggin together with lower concentrations of IGF-I results in a greater percentage of cells that differentiate into oligodendrocytes compared with IGF-I alone, suggesting that IGF-I induction of oligodendrocyte differentiation involves the activation of BMP antagonists, such as Noggin. This finding, together with the observation that there is an upregulation of Noggin and Smad6, 7, further reinforces the role of IGF-I in the inhibition of BMP signaling to promote oligodendrocyte differentiation. A proposed model is shown in FIG. 8. BMP signaling has been shown to alter the fate of neural progenitor cells by stimulating astroglial differentiation while inhibiting neuronal and oligodendroglial differentiation (Mabie et al., 1999; Mehler et al., 1997; Nakashima et al., 2001) (FIG. 8A). IGFs (IGF-I, IGF-II) and/or insulin activate IGF-I receptors located on multipotent neural progenitor cells, which leads to the upregulation of BMP antagonists, such as Noggin and Smad6, 7. Since Noggin and Smad6, 7 inhibit BMP signaling, the net effects of IGF signaling are a decrease or absence of astrocyte differentiation and an increase in neuronal and oligodendroglial differentiation (FIG. 8B). Future studies are needed to determined if all of IGF-instructive effects on oligodendrocyte differentiation occurs through an inhibition of BMP signaling, or if IGFs can directly promote oligodendroglial lineage commitment in a Noggin/Smad6, 7-independent manner.

Materials and Methods

Cell Culture and in vitro Differentiation Analysis

The neural progenitor cells isolated from the hippocampus of female Fischer 344 used in this study have been characterized previously (Gage et al., 1995; Palmer et al., 1997). The whole brain-derived neural stem cells from PIO ICR mice were isolated and cultured according to the methods as described, with slight modifications. Cells were cultured as previously described (Gage et al., 1995; Ray et al., 1993). Cells between passages 10 and 20 were used for in vitro differentiation analyses. To induce differentiation, cells were plated into 4-well chamber slides at a density of 55,000-75,000 cells per well and were allowed to proliferate in N2-supplemented (Invitrogen) DMEM:F12 (Omega Scientific) medium containing 20 ng/ml FGF-2 (PeproTech, Inc.) for 24 hours. FGF-2 was then withdrawn and cells were subsequently treated with differentiation media. Differentiation conditions were either N2 medium with 1 μM RA (Sigma) and 1% FBS (Omega Scientific) for 4 days (mixed); 1 μM RA and 5 μM FSK (Sigma) for 4 days (neuronal), or 50 ng/ml LIF (Chemicon International Inc.) and 50 ng/ml BMP2 (R&D Systems) for 6 days (astrocytic) (Nakashima et al., 1999). For IGF-induction experiments, cells were trypsinized, washed with 1× PBS, and plated into insulin-free N2 medium. Either human recombinant IGF-I or IGF-II (500 ng/ml; R&D Systems) or insulin (500 ng/ml; Sigma) was added for 4 days, except when indicated otherwise. In some cultures, BrdU (2.5 μM, Sigma) was added to label dividing cells, and Q-VD-OPh (2 μM, Enzyme Systems Products) was added to prevent apoptosis.

Immunocytochemistry and in vitro Quantification

Cells were fixed with 4% paraformaldehyde, followed by immunocytochemical staining as described (Palmer et al., 1999). Labeled cells were visualized using a Nikon E800 upright microscope or a Nikon E600 inverted microscope (Nikon Inc.) and a Spot RT CCD camera (Diagnostic Instruments Inc.). 4′,6-diamidino-2-phenylindole (DAPI) was used to identify individual cells. Quantification of cell phenotypes was with StereoInvestigator (MicroBrightfield Inc.); for each marker, 500-1000 cells were sampled systematically from standardized fields at 40×magnification. For quantification of live/dead cells, images were taken of cultures live-stained with propidum iodide (1 μg/ml; Molecular Probes) and Hoescht 33342 (1 μg/ml; Sigma) at 10×magnification, and cells were counted using Image-Pro (Media Cybernetics). The following primary antibodies were used: rabbit anti-Tuj1 (1:7500;Covance), mouse anti-RIP (1:25-1:75; Hybridoma Bank); guinea pig anti-GFAP (1:500-1:2500; Advanced Immunochemical, Inc.); rabbit anti-MBP (1:500; Chemicon International Inc.), rat anti-BrdU (1:400; Accurate). Secondary antibodies were all from Jackson ImmunoResearch and used at 1:250 dilution. The detection of BrdU in cultured cells required treatment in 2 N HCL at 37° C. for 30 min (Palmer et al., 1999). All experiments were independently replicated at least 3 times.

IGF-I Overexpression in vivo and Quantification

cDNA encoding the human IGF-I gene was cloned into a recombinant AAV vector and the virus was prepared as previously described (Kaspar et al., 2002). Expression of IGF-I and \overline{β}gal (control AAV) was first confirmed in human embryonic kidney (HEK-293) cells by RT-PCR and Western analysis and subsequently in the hippocampus by RT-PCR analysis (unpublished data). Recombinant AAV-IGF-I (rAAV-IGF-I) or AAV-\overline{β}gal (rAAV-\overline{β}gal) was stereotaxically injected into the hippocampus (anteroposterior axis, −4.0 mm; mediolateral axis, +2.0 mm; dorsoventral axis, −3.0 from skull, with nose bar at 3.3 mm up) of anesthetized female Fischer-344 rats (150-175 g; n=6). After 4 weeks to allow for virus processing and transgene expression, animals were sacrificed. Animals were perfused with 4% paraformaldehyde and the brains were excised, stored in fixative overnight, and transferred to 30% sucrose. Coronal sections (40 μm) were cut on a sliding microtome and sections were processed for standard immunohistochemical staining as previously described (Gage et al., 1995). Sections were triple labeled with mouse anti-RIP (1:50), rabbit anti-MBP (1:500) and guinea pig anti-GFAP (1:1000). In some cases, sections were labeled with mouse anti-GST-π(1:100; PharMingen). Images were acquired using a Bio-Rad Radiance 2100 confocal microscope and a Nikon TE2000 inverted microscope equipped with a 40× NA1.3 Plan Fluor objective lens. Images were post-processed using Adobe PhotoShop.

All comparative analyses were focused on the hilus of the injected side on matched hippocampal sections. For RIP or MBP staining, immunofluorescence average pixel intensity of equivalent sized fields (4 from each animal) in control and experimental groups was determined from digital images using Adobe Photoshop. To quantify the number of oligodendrocytes, sections were immunostained with GST-π. Only immunolabeled cells with a clear DAPI-positive nucleus were scored. For quantification of GST-π cells, area counts of the hilus within the dentate gyrus of the hippocampus were performed. In each section, 3 adjacent fields were sampled. Cells in the uppermost focal plane were ignored and we focused through the thickness of the section to avoid errors caused by oversampling. The results are expressed as the average number of cells per section.

Quantitative Real Time PCR Analysis

Total RNA was isolated from cell cultures using RNeasy columns (Qiagen). For real-time quantitative PCR, reactions were carried out essentially as described (Zhao et al., 2001). The relative amount of the tested message was normalized to the level of the internal control message, GAPDH. Furthermore, independent experiments were performed with a different internal control message, P-actin, and showed similar results. Primer sequences are available upon request.

Statistics

Results were analyzed for statistical significance using Student's t test or by analysis of variance (ANOVA), and all error bars (except in FIG. 3, D and E) are expressed as standard deviations. Post-hoc analysis was done using Bonferonni corrected planned comparison.

### References

Aberg, M. A., N. D. Aberg, H. Hedbacker, J. Oscarsson, and P. S. Eriksson. 2000. Peripheral infusion of IGF-I selectively induces neurogenesis in the adult rat hippocampus. J Neurosci. 20:2896-903.
Akiyama, K., S. Ichinose, A. Omori, Y. Sakurai, and H. Asou. 2002. Study of expression of myelin basic proteins (MBPs) in developing rat brain using a novel antibody reacting with four major isoforms of MBP. J Neurosci Res. 68:19-28.
Arsenijevic, Y., and S. Weiss. 1998. Insulin-like growth factor-I is a differentiation factor for postmitotic CNS stem cell-derived neuronal precursors: distinct actions from those of brain-derived neurotrophic factor. J Neurosci. 18:2118-28.
Arsenijevic, Y., S. Weiss, B. Schneider, and P. Aebischer. 2001. Insulin-like growth factor-I is necessary for neural stem cell proliferation and demonstrates distinct actions of epidermal growth factor and fibroblast growth factor-2. J Neurosci. 21:7194-202.
Baker, J., J. P. Liu, E. J. Robertson, and A. Efstratiadis. 1993. Role of insulin-like growth factors in embryonic and postnatal growth. Cell. 75:73-82.
Balemans, W., and W. Van Hul. 2002. Extracellular regulation of BMP signaling in vertebrates: a cocktail of modulators. Dev Biol. 250:231-50.
Barres, B. A., R. Schmid, M. Sendnter, and M. C. Raff. 1993. Multiple extracellular signals are required for long-term oligodendrocyte survival. Development. 118:283-95.
Beck, K. D., L. Powell-Braxton, H. R. Widmer, J. Valverde, and F. Hefti. 1995. Igf1 gene disruption results in reduced brain size, CNS hypomyelination, and loss of hippocampal granule and striatal parvalbumin-containing neurons. Neuron. 14:717-30.
Carson, M. J., R. R. Behringer, R. L. Brinster, and F. A. McMorris. 1993. Insulin-like growth factor I increases brain growth and central nervous system myelination in transgenic mice. Neuron. 10:729-40.
Caserta, T. M., A. N. Smith, A. D. Gultice, M. A. Reedy, and T. L. Brown. 2003. Q-VD-OPh, a broad spectrum caspase inhibitor with potent antiapoptotic properties. Apoptosis. 8:345-52.
Christian, J. L., and T. Nakayama. 1999. Can't get no SMADisfaction: Smad proteins as positive and negative regulators of TGF-beta family signals. Bioessays. 21:382-90.
D'Ercole, A. J., P. Ye, A. S. Calikoglu, and G. Gutierrez-Ospina. 1996. The role of the insulin-like growth factors in the central nervous system. Mol Neurobiol. 13:227-55.
Friedman, B., S. Hockfield, J. A. Black, K. A. Woodruff, and S. G. Waxman. 1989. In situ demonstration of mature oligodendrocytes and their processes: an immunocytochemical study with a new monoclonal antibody, rip. Glia. 2:380-90.
Gage, F. H. 2000. Mammalian neural stem cells. Science. 287:1433-8.
Gage, F. H., P. W. Coates, T. D. Palmer, H. G. Kuhn, L. J. Fisher, J. 0. Suhonen, D. A. Peterson, S. T. Suhr, and J. Ray. 1995. Survival and differentiation of adult neuronal progenitor cells transplanted to the adult brain. Proc Natl Acad Sci USA. 92:11879-83.
Gross, R. E., M. F. Mehler, P. C. Mabie, Z. Zang, L. Santschi, and J. A. Kessler. 1996. Bone morphogenetic proteins promote astroglial lineage commitment by mammalian subventricular zone progenitor cells. Neuron. 17:595-606.
Homer, P. J., A. E. Power, G. Kempermann, H. G. Kuhn, T. D. Palmer, J. Winkler, L. J. Thal, and F. H. Gage. 2000. Proliferation and differentiation of progenitor cells throughout the intact adult rat spinal cord. J Neurosci. 20:2218-28.
Kaspar, B. K., B. Vissel, T. Bengoechea, S. Crone, L. Randolph-Moore, R. Muller, E. P. Brandon, D. Schaffer, I. M. Verma, K. F. Lee, S. F. Heinemann, and F. H. Gage. 2002. Adeno-associated virus effectively mediates conditional gene modification in the brain. Proc Natl Acad Sci USA. 99:2320-5.
Kondo, T., and M. Raff. 2000. Oligodendrocyte precursor cells reprogrammed to become multipotential CNS stem cells. Science. 289:1754-7.
Kornack, D. R., and P. Rakic. 2001. Cell proliferation without neurogenesis in adult primate neocortex. Science. 294:2127-30.
LeRoith, D., H. Werner, T. N. Faria, H. Kato, M. Adamo, and C. T. Roberts, Jr. 1993. Insulin-like growth factor receptors. Implications for nervous system function. Ann N Y Acad Sci. 692:22-32.
Lim, D. A., A. D. Tramontin, J. M. Trevejo, D. G. Herrera, J. M. Garcia-Verdugo, and A. Alvarez-Buylla. 2000. Noggin antagonizes BMP signaling to create a niche for adult neurogenesis. Neuron. 28:713-26.
Lois, C., and A. Alvarez-Buylla. 1993. Proliferating subventricular zone cells in the adult mammalian forebrain can differentiate into neurons and glia. Proc Natl Acad Sci USA. 90:2074-7.
Mabie, P. C., M. F. Mehler, and J. A. Kessler. 1999. Multiple roles of bone morphogenetic protein signaling in the regulation of cortical cell number and phenotype. J Neurosci. 19:7077-88.
Mason, J. L., K. Suzuki, D. D. Chaplin, and G. K. Matsushima. 2001. Interleukin-1beta promotes repair of the CNS. J Neurosci. 21:7046-52.
McMorris, F. A., and M. Dubois-Dalcq. 1988. Insulin-like growth factor I promotes cell proliferation and oligodendroglial commitment in rat glial progenitor cells developing in vitro. J Neurosci Res. 21:199-209.
McMorris, F. A., T. M. Smith, S. DeSalvo, and R. W. Furlanetto. 1986. Insulin-like growth factor I/somatomedin C: a potent inducer of oligodendrocyte development. Proc Natl Acad Sci USA. 83:822-6.
Mehler, M. F., P. C. Mabie, D. Zhang, and J. A. Kessler. 1997. Bone morphogenetic proteins in the nervous system. Trends Neurosci. 20:309-17.
Mehler, M. F., P. C. Mabie, G. Zhu, S. Gokhan, and J. A. Kessler. 2000. Developmental changes in progenitor cell responsiveness to bone morphogenetic proteins differentially modulate progressive CNS lineage fate. Dev Neurosci. 22:74-85.
Mekki-Dauriac, S., E. Agius, P. Kan, and P. Cochard. 2002. Bone morphogenetic proteins negatively control oligodendrocyte precursor specification in the chick spinal cord. Development. 129:5117-30.
Nakashima, K., T. Takizawa, W. Ochiai, M. Yanagisawa, T. Hisatsune, M. Nakafuku, K. Miyazono, T. Kishimoto, R. Kageyama, and T. Taga. 2001. BMP2-mediated alteration in the developmental pathway of fetal mouse brain cells from neurogenesis to astrocytogenesis. Proc Natl Acad Sci USA. 98:5868-73.
Nakashima, K., M. Yanagisawa, H. Arakawa, N. Kimura, T. Hisatsune, M. Kawabata, K. Miyazono, and T. Taga. 1999. Synergistic signaling in fetal brain by STAT3-Smad1 complex bridged by p300. Science. 284:479-82.
Palmer, T. D., E. A. Markakis, A. R. Willhoite, F. Safar, and F. H. Gage. 1999. Fibroblast growth factor-2 activates a latent neurogenic program in neural stem cells from diverse regions of the adult CNS. J Neurosci. 19:8487-97.
Palmer, T. D., J. Takahashi, and F. H. Gage. 1997. The adult rat hippocampus contains primordial neural stem cells. Mol Cell Neurosci. 8:389-404.
Rakic, P. 2002. Adult neurogenesis in mammals: an identity crisis. J Neurosci. 22:614-8.
Ray, J., D. A. Peterson, M. Schinstine, and F. H. Gage. 1993. Proliferation, differentiation, and long-term culture of primary hippocampal neurons. Proc Natl Acad Sci USA. 90:3602-6.
Reynolds, B. A., and S. Weiss. 1992. Generation of neurons and astrocytes from isolated cells of the adult mammalian central nervous system. Science. 255:1707-10.
Shihabuddin, L. S., P. J. Homer, J. Ray, and F. H. Gage. 2000. Adult spinal cord stem cells generate neurons after transplantation in the adult dentate gyrus. J Neurosci. 20:8727-35.
Shihabuddin, L. S., J. Ray, and F. H. Gage. 1997. FGF-2 is sufficient to isolate progenitors found in the adult mammalian spinal cord. Exp Neurol. 148:577-86.
Song, H., C. F. Stevens, and F. H. Gage. 2002. Astroglia induce neurogenesis from adult neural stem cells. Nature. 417:39-44.
Suhonen, J. O., D. A. Peterson, J. Ray, and F. H. Gage. 1996. Differentiation of adult hippocampus-derived progenitors into olfactory neurons in vivo. Nature. 383:624-7.
Takahashi, J., T. D. Palmer, and F. H. Gage. 1999. Retinoic acid and neurotrophins collaborate to regulate neurogenesis in adult-derived neural stem cell cultures. J Neurobiol. 38:65-81.
Tanigaki, K., F. Nogaki, J. Takahashi, K. Tashiro, H. Kurooka, and T. Honjo. 2001. Notch1 and Notch3 instructively restrict bFGF-responsive multipotent neural progenitor cells to an astroglial fate. Neuron. 29:45-55.
Tansey, F. A., and W. Cammer. 1991. A pi form of glutathione-S-transferase is a myelin- and oligodendrocyte-associated enzyme in mouse brain. J Neurochem. 57:95-102.
Vincent, A. M., and E. L. Feldman. 2002. Control of cell survival by IGF signaling pathways. Growth Horm IGF Res. 12:193-7.
Ye, P., J. Carson, and A. J. D'Ercole. 1995. In vivo actions of insulin-like growth factor-I (IGF-I) on brain myelination: studies of IGF-I and IGF binding protein-1 (IGFBP-1) transgenic mice. J Neurosci. 15:7344-56.
Ye, P., L. Li, R. G. Richards, R. P. DiAugustine, and A. J. D'Ercole. 2002. Myelination is altered in insulin-like growth factor-I null mutant mice. J Neurosci. 22:6041-51.
Zhao, X., E. S. Lein, A. He, S. C. Smith, C. Aston, and F. H. Gage. 2001. Transcriptional profiling reveals strict boundaries between hippocampal subregions. J Comp Neurol. 441:187-96.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of inducing differentiation of mammalian multipotent neural stem cells into oligodendrocytes, the method comprising the step of:
contacting a population of mammalian multipotent neural stem cells with a physiologically acceptable composition comprising no more than one type of a recombinant growth factor, said recombinant growth factor consisting of recombinant IGF-1 in an amount sufficient to induce said mammalian multipotent neural stem cells to differentiate into oligodendrocytes;
wherein said mammalian multipotent neural stem cells produce a differentiated cell population comprising at least about 10% oligodendrocytes.

2. The method of claim 1, wherein said mammalian multipotent neural stem cells are mammalian adult CNS multipotent neural stem cells.

3. The method of claim 1, wherein said mammalian multipotent neural stem cells are human cells.

4. The method of claim 1,wherein said differentiated cell population comprises at least about 40% oligodendrocytes.

5. The method of claim 1, wherein at least about 40% of cells in said differentiated cell population are RIP positive.

6. The method of claim 1,wherein at least about 40% of cells in said differentiated cell population have morphological features of oligodendrocytes.

7. The method of claim 1, wherein said mammalian multipotent neural stem cells are from the central nervous system.

8. The method of claim 1, wherein said mammalian multipotent neural stem cells are from the adult central nervous system.

9. The method of claim 1, wherein said mammalian multipotent neural stem cells are from mammalian neural tissue selected from the group consisting of hippocampal tissue, subventricular tissue, cerebral cortex tissue, cerebellum tissue, midbrain tissue, brainstem tissue, spinal cord tissue, ventricular tissue, whole brain tissue, and combinations thereof.

10. The method of claim 1, wherein said recombinant IGF-1 is exogenously added to said mammalian multipotent neural stem cells.

11. The method of claim 1, wherein said recombinant IGF-1 is expressed by genetically modified cells.

12. The method of claim 11, wherein said genetically modified cells are mammalian adult CNS multipotent neural stem cells.

13. A method of inducing in vitro differentiation of mammalian multipotent neural stem cells into oligodendrocytes, the method comprising the steps of:
(a) obtaining a population of mammalian multipotent neural stem cells;
(b) preparing a culture medium comprising no more than one type of a recombinant growth factor, said recombinant growth factor consisting of recombinant IGF-1 in an amount sufficient to induce said mammalian multipotent neural stem cells to differentiate into oligodendrocytes;
(c) combining said mammalian multipotent neural stem cells with said culture medium; and
(d) culturing said mammalian multipotent neural stem cells under conditions that produce a differentiated cell population comprising at least about 10% oligodendrocytes.

14. The method of claim 13, wherein said mammalian multipotent neural stem cells are mammalian adult CNS multipotent neural stem cells.

15. The method of claim 13, wherein said mammalian multipotent neural stem cells are human cells.

16. The method of claim 13, wherein said differentiated cell population comprises at least about 40% oligodendrocytes.

17. The method of claim 13, wherein at least about 40% of the cells in said differentiated cell population are RIP positive cells.

18. The method of claim 13, wherein at least 40% of the cells in said differentiated cell population have morphological features of oligodendrocytes.

19. The method of claim 13, wherein said recombinant IGF-1 is present in said cell culture at a concentration of about 5 ng/ml to about 5 μg/ml.

20. The method of claim 13, wherein said recombinant IGF-1 is present in said cell culture at a concentration of about 50 ng/ml to about 1 μg/ml.

21. The method of claim 13, wherein said mammalian multipotent neural stem cells are from the central nervous system.

22. The method of claim 13, wherein said mammalian multipotent neural stem cells are from the adult central nervous system.

23. The method of claim 13, wherein said mammalian multipotent neural stem cells are from mammalian neural tissue selected from the group consisting of hippocampal tissue, subventricular tissue, cerebral cortex tissue, cerebellum tissue, midbrain tissue, brainstem tissue, spinal cord tissue, ventricular tissue, whole brain tissue, and combinations thereof.

24. The method of claim 13, wherein said culture medium prepared in step (b) is defined.

25. The method of claim 13, wherein said culture medium prepared in step (b) is serum free.

26. The method of claim 13, wherein said recombinant IGF-1 is exogenously added to said culture medium.

27. The method of claim 13, wherein said recombinant IGF-1 is expressed by genetically modified cells in said culture medium.

28. The method of claim 27, wherein said genetically modified cells are mammalian adult CNS multipotent neural stem cells.

29. A method of inducing ex vivo differentiation of mammalian multipotent neural stem cells into oligodendrocytes, the method comprising the steps of:
(a) obtaining a population of mammalian multipotent neural stem cells from a patient;
(b) preparing a culture medium comprising no more than one type of a recombinant growth factor, said recombinant growth factor consisting of recombinant IGF-1 in an amount sufficient to induce said mammalian multipotent neural stem cells to differentiate into oligodendrocytes;
(c) combining said mammalian multipotent neural stem cells with said culture medium;
(d) culturing said mammalian multipotent neural stem cells under conditions that produce a differentiated cell population comprising at least about 10% oligodendrocytes; and
(e) administering the differentiated cell population to the patient.

30. A method of inducing in vivo differentiation of adult mammalian multipotent neural stem cells into oligodendrocytes, the method comprising the steps of:
(a) administering a physiologically acceptable composition to the hippocampal zone of a subject, the composition comprising no more than one type of a recombinant growth factor, said recombinant growth factor consisting of recombinant IGF-1 in an amount sufficient to induce said adult mammalian multipotent neural stem cells to differentiate into oligodendrocytes.

31. A method of inducing in vivo differentiation of adult mammalian multipotent neural stem cells into oligodendrocytes, the method comprising the steps of:
(a) administering a physiologically acceptable composition to the hippocampal zone of a subject, the composition comprising a nucleic acid encoding no more than one type of a recombinant growth factor, said recombinant growth factor consisting of recombinant IGF-1, wherein the recombinant IGF-1 is produced by the nucleic acid in cells and secreted in an amount sufficient to induce said adult mammalian multipotent neural stem cells to differentiate into oligodendrocytes.

32. A method of screening for a compound that modulates the differentiation of mammalian multipotent neural stem cells into oligodendrocytes, the method comprising the steps of:
(a) obtaining mammalian multipotent neural stem cells;
(b) preparing a culture medium comprising a recombinant growth factor, said recombinant growth factor consisting of recombinant IGF-1 in an amount sufficient to induce said mammalian multipotent neural stem cells to differentiate into oligodendrocytes;
(c) combining said mammalian multipotent neural stem cells with said culture medium and said compound;
(d) culturing said mammalian multipotent neural stem cells under conditions that, in the absence of said compound, produce a differentiated cell population comprising at least about 10% oligodendrocytes; and
(e) determining the effect of the compound on the differentiation of said mammalian multipotent neural stem cells into oligodendrocytes.

33. The method of claim 32, wherein said mammalian multipotent neural stem cells are mammalian adult CNS multipotent neural stem cells.

34. The method of claim 32, wherein said mammalian multipotent neural stem cells are human cells.

35. The method of claim 32, wherein said recombinant IGF-1 is present in said cell culture at a concentration of about 5 ng/ml to about 5 μg/ml.

36. The method of claim 32, wherein said recombinant IGF-1 is present in said cell culture at a concentration of about 50 ng/ml to about 1 μg/ml.

37. The method of claim 32, wherein said mammalian multipotent neural stem cells are from the central nervous system.

38. The method of claim 32, wherein said mammalian multipotent neural stem cells are from the adult central nervous system.

39. The method of claim 32, wherein said mammalian multipotent neural stem cells are from mammalian neural tissue selected from the group consisting of hippocampal tissue, subventricular tissue, cerebral cortex tissue, cerebellum tissue, midbrain tissue, brainstem tissue, spinal cord tissue, ventricular tissue, whole brain tissue, and combinations thereof.

40. The method of claim 32, wherein said culture medium prepared in step (b) is defined.

41. The method of claim 32, wherein said culture medium prepared in step (b) is serum free.

42. The method of claim 32, wherein said recombinant IGF-1 is exogenously added to said culture medium.

43. The method of claim 32, wherein said recombinant IGF-1 is expressed by genetically modified cells in said culture medium.

44. The method of claim 43, wherein said genetically modified cells are mammalian adult CNS multipotent neural stem cells.

45. The method of claim 32, wherein the compound is a small organic molecule.

46. A method of treating a patient who has a neurological deficit, the method comprising the step of:
contacting a population of mammalian multipotent neural stem cells with a physiologically acceptable composition comprising a recombinant growth factor, said recombinant growth factor consisting of recombinant IGF-1 in an amount sufficient to induce said mammalian multipotent neural stem cells to differentiate into oligodendrocytes;
wherein said mammalian multipotent neural stem cells produce a differentiated cell population comprising at least about 10% oligodendrocytes.

47. The method of claim 46, wherein the neurological deficit is caused by a neurodegenerative disease, age-related dementia, schizophrenia, bipolar disorder, major depression, a traumatic brain injury, a neurotoxic injury, ischemia, a developmental disorder, a disorder affecting vision, an injury or disease of the spinal cord, a demyelinating disease, an autoimmune disease, an infection, or an inflammatory disease.

48. The method of claim 46, wherein the ischemia is caused by a stroke.

49. The method of claim 46, wherein the neurodegenerative disease is selected from the group consisting of Alzheimer's disease, multiple sclerosis (MS), Huntington's disease, amyotrophic lateral sclerosis, and Parkinson's disease.

50. The method of claim 46, wherein said mammalian multipotent neural stem cells are contacted with exogenous recombinant IGF-1.

51. The method of claim 46, wherein said recombinant IGF-1 is expressed by genetically modified cells.

52. The method of claim 51, wherein said genetically modified cells are mammalian adult CNS multipotent neural stem cells.

53. The method of claim 46, wherein said mammalian multipotent neural stem cells are human adult CNS multipotent neural stem cells.

54. The method of claim 46, wherein said mammalian multipotent neural stem cells are human cells.

55. The method of claim 46, wherein said mammalian multipotent neural stem cells are from the central nervous system.

56. The method of claim 46, wherein said mammalian multipotent neural stem cells are from the adult central nervous system.

57. The method of claim 46, wherein said mammalian multipotent neural stem cells are from mammalian neural tissue selected from the group consisting of hippocampal tissue, subventricular tissue, cerebral cortex tissue, cerebellum tissue, midbrain tissue, brainstem tissue, spinal cord tissue, ventricular tissue, whole brain tissue, and combinations thereof.

58. A stable in vitro culture of oligodendrocytes, said culture obtained by differentiating mammalian multipotent neural stem cells by contacting said mammalian multipotent neural stem cells with a physiologically acceptable composition comprising a recombinant growth factor, said recombinant growth factor consisting of recombinant IGF-1 in an amount sufficient to induce said mammalian multipotent neural stem cells to differentiate into oligodendrocytes, said culture comprising at least about 10% oligodendrocytes.

59. The cell population of claim 58, wherein at least 40% of cells of said culture have morphological features of oligodendrocytes.

60. The cell population of claim 58, wherein at least about 40% of cells of said culture are RIP positive.

61. The cell population of claim 58, wherein at least 40% of cells of said culture have morphological features of oligodendrocytes.

62. A method of inducing in vitro differentiation of mammalian multipotent neural stem cells into oligodendrocytes, the method comprising the steps of:
(a) obtaining a population of mammalian multipotent neural stem cells;
(b) preparing a culture medium comprising a recombinant growth factor, said recombinant growth factor consisting of recombinant IGF-1, recombinant IGF-2 or recombinant insulin, in an amount sufficient to induce said mammalian multipotent neural stem cells to differentiate into oligodendrocytes;
(c) combining said mammalian multipotent neural stem cells with said culture medium; and
(d) culturing said mammalian multipotent neural stem cells under conditions that produce a cell culture comprising at least about 10% oligodendrocytes.
